SEQUENCE LISTING (2 sequences)

SEQ ID NO: 1 - protein, 116 aa, Artificial Sequence
  Synthetic construct. “Variable region of the PDL humanized anti-Tac antibody heavy chain.”
QVQLVQSGAEVKKPGSSVKVSCKASGYTFTSYRMHWVRQAPGQGLEWIGYINPSTGYTEYNQKFKDKATITADESTNTAYMELSSLRSEDTAVYYCARGGGVFDYWGQGTLVTVSS

SEQ ID NO: 2 - protein, 106 aa, Artificial Sequence
  Synthetic construct. “Variable region of the PDL humanized anti-Tac antibody light chain.”
DIQMTQSPSTLSASVGDRVTITCSASSSISYMHWYQQKPGKAPKLLIYTTSNLASGVPARFSGSGSGTEFTLTISSLQPDDFATYYCHQRSTYPLTFGQGTKVEVK

(19) United States
(12) Patent Application Publication
(10) Pub. No.: US 2011/0053209 A1
(43) Pub. Date: Mar. 3, 2011

(54) USE OF AN IMMUNOREGULATORY NK CELL POPULATION FOR MONITORING THE EFFICACY OF ANTI-IL-2R ANTIBODIES IN MULTIPLE SCLEROSIS PATIENTS

(75) Inventor: James Peter Sheridan, III, Mountain View, CA (US)

(73) Assignee: FACET BIOTECH CORPORATION, Redwood City, CA (US)

(21) Appl. No.: 12/872,886

(22) Filed: Aug. 31, 2010

Related U.S. Application Data
(60) Provisional application No. 61/238,353, filed on Aug. 31, 2009.

(51) Int. Cl. C12Q 1/02

(52) U.S. Cl. 435/29

(57) ABSTRACT

The use of CD56bright NK cell counts as a biomarker for the efficacy of anti-IL-2R antibody treatment in patients diagnosed with multiple sclerosis.

### 1. CROSS REFERENCE TO RELATED APPLICATIONS

This application claims benefit under 35 U.S.C. §119(e) to application Ser. No. 61/238,353 filed Aug. 31, 2009, the entire contents of which are incorporated herein by reference.

### 2. STATEMENT REGARDING FEDERALLY SPONSORED RESEARCH

Not applicable.

### 3. BACKGROUND

The goal of multiple sclerosis (MS) treatment is to prevent permanent disabilities and delay disease progression. A shorter-term therapeutic goal focuses on reducing the relapse rate. IFN-beta is the most commonly used chronic maintenance agent for treating MS. Drawbacks to IFN-beta treatment include inadequate response in at least 30% of patients following initiation of IFN-beta therapy (Bertolotto, et al., 2002, J Neurosurg Psychiatry, 73(2):148-153; and Durelli, 2004, J Neurol., 251 Suppl4:IV13-IV24), and induction of anti-interferon antibodies in 7% to 42% of patients (Sorensen, 2003, Lancet, 362:1184-91). Although other agents are used to treat MS, including corticosteroids, glatiramer acetate, mitoxantrone, and natalizumab, these agents are only partially effective in managing clinical relapses, and some carry significant safety risks.

Because the agents currently being used to treat MS are not completely effective in managing the disease, it is desirable to identify and clinically validate markers that can be used to monitor the clinical response in an individual diagnosed with MS to a therapeutic agent as means for optimizing and/or changing the treatment regime for that individual.

### 3. SUMMARY

One agent that has shown promise for treating MS in clinical trials is daclizumab. Daclizumab was evaluated as a treatment for MS in a Phase 2, randomized, double-blinded, placebo-controlled, multi-center, dose-ranging study (the CHOICE study). At the end of the 24-week dosing period, compared to IFN-beta placebo, there was a 25% reduction in new or enlarged gadolinium contrast enhancing lesions (Gd-CEL) as detected by magnetic resonance imaging (MRI) in the daclizumab 1 mg/kg group and a 72% reduction in the daclizumab 2 mg/kg group (FIG. 1). Both daclizumab regimes were associated with an approximate 35% reduction in annualized relapse rate at 24 weeks (Montalban, X. et al., Multiple Sclerosis, 13: S18-S18 Suppl. 2 OCT 2007; and, Kaufman, M. D., et. a., Neurology, 70 (11): A220-A220 Suppl. 1 Mar. 11, 2008).

Patient blood samples were collected throughout the CHOICE study and analyzed to determine levels of immune cell subsets and associated activation markers prior to, during, and after treatment with daclizumab. A rapid expansion of CD56^{bright} NK cells was detected in MS patients treated with daclizumab. This expansion was dose-dependent and detectable within 14 days following the first dose of daclizumab in the 1 mg/kg and 2 mg/kg dosing groups. The exposure-dependent expansion in CD56^{bright} NK cell numbers was consistent with the exposure-dependent reductions in new or enlarged Gd-CEL (see, e.g., FIG. 5).

These results indicate that rapid expansion in CD56^{bright} NK cell levels can be used as a biomarker of clinical response to daclizumab in a patient diagnosed with MS. Accordingly, the methods described herein disclose the use of CD56^{bright} NK cell levels to monitor the efficacy of daclizumab in a patient diagnosed with MS. In some embodiments, the method comprises determining the level of CD56^{bright} NK cells in a patient diagnosed with MS within 14 days after the first dose of daclizumab, wherein an increase in the level of CD56^{bright} NK cells indicates that daclizumab is effective in ameliorating at least one symptom of MS in the treated patient. Biological samples from the treated patient can be collected at one or more times prior to, during, and/or after treatment with daclizumab.

Symptoms of MS that can be stabilized or improved using the methods described herein include, but are not limited to, reducing the relapse rate, stabilizing or reducing the rate of disability progression as measured by standard scores such as the Expanded Disability Status Scale (EDSS) score, decreasing the number of new or enlarged Gd-CEL, and/or decreasing the number of new or enlarged T2 MRI lesions. The subject being treated can have relapsing forms of MS, including relapsing/remitting MS, secondary progressive MS, progressive relapsing MS, or worsening relapsing MS. In addition to daclizumab, other IL-2R antibodies, such as monoclonal antibodies, chimeric antibodies, humanized antibodies, or fully human antibodies that specifically bind to the alpha or p55 (Tac) chain of the IL-2 receptor can be used in the methods described herein.

It is to be understood that both the foregoing general description and the following detailed description are exemplary and explanatory only and are not restrictive of the compositions and methods described herein. In this application, the use of the singular includes the plural unless specifically state otherwise. Also, the use of “or” means “and/or” unless state otherwise. Similarly, “comprise,” “comprises,” “comprising,” “include,” “includes” and “including” are not intended to be limiting.

### 4. BRIEF DESCRIPTION OF THE FIGURES

FIG. 1 is a bar graph depicting data demonstrating reduction in Gd-CEL at the end of a 24 week dosing period with daclizumab;

FIG. 2A-FIG. 2C are Lowess plots of CD56^{bright} NK cell counts over time in the placebo group (FIG. 2A), the low dose (1 mg/kg) daclizumab treatment group (FIG. 2B), and the high dose (2 mg/kg) daclizumab treatment group (FIG. 2C);

FIG. 3 is a line graph depicting a statistically significant (p=0.0022) positive linear relationship between each individual's steady state exposure to daclizumab and the change from baseline level in CD56^{bright} NK cell counts when each was expressed as an area under the curve (AUC) measurement;

FIG. 4 is a mixed-effects linear regression model illustrating the dose-dependent expansion of CD56^{bright} NK cells in daclizumab low dose and high dose treatment groups; and;

FIG. 5 is a bar graph depicting reductions in new or enlarged Gd-CEL by quartile after ranking all daclizumab treated subjects according to their increase in CD56^{bright} NK cell counts.

### 5. DETAILED DESCRIPTION

### 5.1 Definitions

As used herein, the following terms are intended to have the following meanings:

The term “antibody” refers to an immunoglobulin molecule that specifically binds to, or is immunologically reactive with, a particular antigen, and includes polyclonal, monoclonal, genetically engineered (e.g., rIgG) and otherwise modified forms of antibodies, including but not limited to chimeric antibodies, humanized antibodies, heteroconjugate antibodies (including, e.g., bispecific antibodies), antigen binding fragments of antibodies, including e.g., Fab′, F(ab′)₂, Fab, Fv, and scFv fragments and multimerici forms of antigen binding fragments, including e.g., diabodies, triabodies and tetrabodies. Moreover, unless otherwise indicated, the term “monoclonal antibody” (mAb) is meant to include both intact molecules, as well as, antibody fragments (such as, for example, Fab and F(ab′)₂ fragments) which are capable of specifically binding to a protein. Fab and F(ab′)₂ fragments lack the Fc fragment of intact antibody, clear more rapidly from the circulation of the animal or plant, and may have less non-specific tissue binding than an intact antibody (Wahl et al., 1983, J. Nucl. Med. 24:316).

The term “scFv” refers to a single chain Fv antibody in which the variable domains of the heavy chain and the light chain from a traditional antibody have been joined to form one chain.

References to “VH” refer to the variable region of an immunoglobulin heavy chain of an antibody, including the heavy chain of an Fv, scFv, or Fab. References to “VL” refer to the variable region of an immunoglobulin light chain, including the light chain of an Fv, scFv, dsFv or Fab. Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrameric glycoproteins of about 150,000 daltons, composed of two identical light (L) chains and two identical heavy (H) chains. Each heavy chain has at one end a variable domain (VH) followed by a number of constant domains. Each light chain has a variable domain at one end (VL) and a constant domain at its other end.

Complementarity determining regions (CDRs) are also known as hypervariable regions both in the light chain and the heavy chain variable domains. The more highly conserved portions of variable domains are called the framework (FR). As is known in the art, the amino acid position/boundary delineating a hypervariable region of an antibody can vary, depending on the context and the various definitions known in the art. Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. The disclosure provides antibodies comprising modifications in these hybrid hypervariable positions. The variable domains of native heavy and light chains each comprise four FR regions, largely by adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the target binding site of antibodies (See Kabat et al., Sequences of Proteins of Immunological Interest (National Institute of Health, Bethesda, Md. 1987). As used herein, numbering of immunoglobulin amino acid residues is done according to the immunoglobulin amino acid residue numbering system of Kabat et al., unless otherwise indicated.

The term “antibody fragment” refers to a portion of a full-length antibody, generally the target binding or variable region. Examples of antibody fragments include Fab, Fab′, F(ab′)2 and Fv fragments. An “Fv” fragment is the minimum antibody fragment which contains a complete target recognition and binding site. This region consists of a dimer of one heavy and one light chain variable domain in a tight, non-covalent association (VH-VL dimer). It is in this configuration that the three CDRs of each variable domain interact to define a target binding site on the surface of the VH-VL dimer. Collectively, the six CDRs confer target binding specificity to the antibody. However, even a single variable domain (or half of an Fv comprising only three CDRs specific for a target) has the ability to recognize and bind target, although at a lower affinity than the entire binding site. “Single-chain Fv” or “scFv” antibody fragments comprise the VH and VL domains of an antibody, wherein these domains are present in a single polypeptide chain. Generally, the Fv polypeptide further comprises a polypeptide linker between the VH and VL domains which enables the scFv to form the desired structure for target binding.

The Fab fragment contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. Fab′ fragments are produced by cleavage of the disulfide bond at the hinge cysteines of the F(ab′)₂ pepsin digestion product. Additional chemical couplings of antibody fragments are known to those of ordinary skill in the art.

“Epitope” or “antigenic determinant” refers to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

The determination of whether two antibodies bind substantially to the same epitope is accomplished using methods known in the art, such as a competition assay. In conducting an antibody competition study between a control antibody (for example, daclizumab) and any test antibody, one may first label the control antibody with a detectable label, such as, biotin, an enzyme, radioactive label, or fluorescent label to enable the subsequent identification. In such an assay, the intensity of bound label is measured in a sample containing the labeled control antibody and the intensity of bound label sample containing the labeled control antibody and the unlabeled test antibody is measured. If the unlabeled test antibody competes with the labeled antibody by binding to an overlapping epitope, the detected label intensity will be decreased relative to the binding in the sample containing only the labeled control antibody. Other methods of determining binding are known in the art.

The term “monoclonal antibody” refers to an antibody that is derived from a single clone, including any eukaryotic, prokaryotic, or phage clone, and not the method by which it is produced. Antibodies immunologically reactive with a particular antigen can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow and Lane, “Antibodies: A Laboratory Manual,” Cold Spring Harbor Laboratory Press, New York (1988); Hammerling et al., in: “Monoclonal Antibodies and T-Cell Hybridomas,” Elsevier, N.Y. (1981), pp. 563 681 (both of which are incorporated herein by reference in their entireties).

A “chimeric antibody” is an immunoglobulin molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. Any of the anti-IL-2R antibodies described herein can be chimeric.

The term “humanized antibody” or “humanized immunoglobulin” refers to an immunoglobulin comprising a human framework, at least one and preferably all complementarity determining regions (CDRs) from a non-human antibody, and in which any constant region present is substantially identical to a human immunoglobulin constant region, i.e., at least about 85%, at least 90%, and at least 95% identical. Hence, all parts of a humanized immunoglobulin, except possibly the CDRs, are substantially identical to corresponding parts of one or more native human immunoglobulin sequences. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. See, e.g., Queen et al., U.S. Pat. Nos. 5,530,101; 5,585,089; 5,693,761; 5,693,762; 6,180,370 (each of which is incorporated by reference in its entirety). Antibodies can be humanized using a variety of techniques known in the art including, for example, CDR-grafting (EP 239,400; PCT publication WO 91/09967; U.S. Pat. Nos. 5,225,539; 5,530,101 and 5,585,089), veneering or resurfacing (EP 592,106; EP 519,596; Padlan, Mol. Immunol., 28:489 498 (1991); Studnicka et al., Prot. Eng. 7:805 814 (1994); Roguska et al., Proc. Natl. Acad. Sci. USA, 91:969 973 (1994), and chain shuffling (U.S. Pat. No. 5,565,332), all of which are hereby incorporated by reference in their entireties. The anti-IL-2R antibodies described herein include humanized antibodies, such as mouse humanized antibodies, fully human antibodies, and mouse antibodies.

An “anti-IL-2R antibody” is an antibody that specifically binds an IL-2 receptor. For example, in some embodiments, an anti-IL-2R antibody binds the high affinity IL-2 receptor (K_{d}˜10 pM). This receptor is a membrane receptor complex consisting of the subunits: IL-2R-alpha (also known as T cell activation (Tac) antigen, CD25, or p55), IL-2R-beta (also known as p75 or CD122), and the cytokine receptor common gamma chain (also known as CD132). In other embodiments, an anti-IL-2R antibody binds the intermediate affinity IL-2 receptor (K_{d}=100 pM), which consists of the p75 subunit and a gamma chain. In other embodiments, an anti-IL-2R antibody binds the low affinity receptor (K_{d}=10 nM), which is formed by p55 alone.

Anti-IL-2R antibodies suitable for use in the methods described herein include monoclonal antibodies, chimeric antibodies, humanized antibodies, or fully human antibodies. Examples of anti-IL-2R antibodies capable of binding Tac (p55) include, but are not limited to, daclizumab, the chimeric antibody basiliximab, BT563 (see Baan et al., Transplant. Proc. 33:224-2246, 2001), and 7G8, and HuMax-TAC (being developed by GenMab). The mik-betal antibody specifically binds the beta chain of human IL-2R. Additional antibodies that specifically bind the IL-2 receptor are known in the art. For example, see U.S. Pat. No. 5,011,684; U.S. Pat. No. 5,152,980; U.S. Pat. No. 5,336,489; U.S. Pat. No. 5,510,105; U.S. Pat. No. 5,571,507; U.S. Pat. No. 5,587,162; U.S. Pat. No. 5,607,675; U.S. Pat. No. 5,674,494; U.S. Pat. No. 5,916,559.

The term “CD56^{bright} NK cell” describes an immune cell that can be characterized by the absence of the CD3 protein on its outer surface, the presence of three fold higher to 10 fold higher levels of the CD56 protein on its outer surface in comparison to other CD56 positive immune cells, and no detectable or very low levels of the CD16 protein.

### 5.2 Detailed Description

MS is an inflammatory/demyelinating disease of the CNS that is one of the leading causes of neurological disability in young adults (Bielekova, B. and Martin, R., 1999, Curr Treat Options Neurol. 1:201-219). The pathogenesis observed in MS patients is, at least in part, attributable to aberrant T-cell activation. Daclizumab is a humanized antibody that binds the IL-2R alpha chain (also known as T cell activation (TAC) antigen, CD25 or p55). CD25 is present at low levels in resting human T cells, but is significantly up-regulated on activated T cells, enabling them to receive a signal through the high-affinity IL-2 receptor (Waldmann, et al., 1998, Int Rev Immunol, 16:205-226). It has been proposed that selective binding of CD25 by daclizumab inhibits IL-2R signal transduction events (Goebel, J., et al., 2000, Transplant Immunol, 8:153-159, Tkaczuk, J., 2001, Transplant Proc, 33:212-213) and blocks T cell activation (Queen, C., et al., 1989, Proc Natl Acad Sci USA, 86:10029-10033).

Bielekova et al., analyzed changes in the level of CD4+ T cells, CD8+ T cells, and CD56^{bright} NK cells in MS patients treated with daclizumab (Bielekova, B., et al., 2006, Proc Natl Acad Sci USA, 103:5941-5946). The level of CD56^{bright} NK cells in MS patients treated with daclizumab was determined at 5.5 months after treatment of the patients with approximately six daclizumab infusions and at 16.5 months, after treatment of the patients with approximately 17 daclizumab infusions (Bielekova, B., et al., 2006, Proc Natl Acad Sci USA, 103:5941-5946). Bielekova et al., reported that daclizumab therapy resulted in an expansion of CD56^{bright} NK cells that correlated strongly with decreases in brain inflammatory activity. The positive correlations between expansion of CD56^{bright} NK cells and contraction of CD4⁺ and CD8⁺ T cell numbers was proposed to support the existence of an immunoregulatory pathway wherein activated CD56^{bright} NK cells control the adaptive immune responses by the negative immunoregulation of activated lymphocytes (Bielekova, B., et al., 2006, Proc Natl Acad Sci USA, 103:5941-5946).

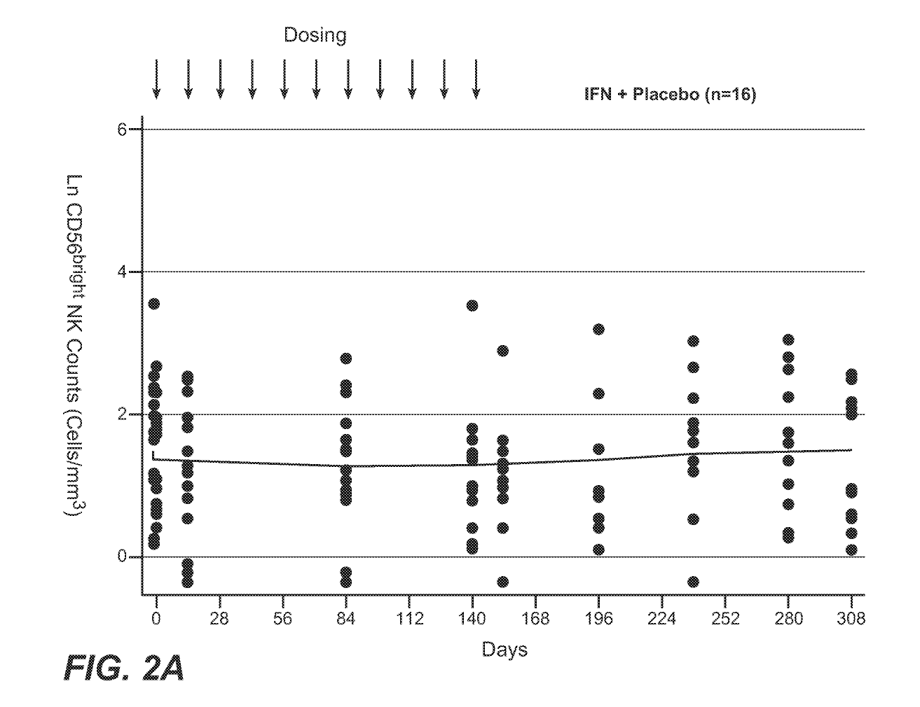

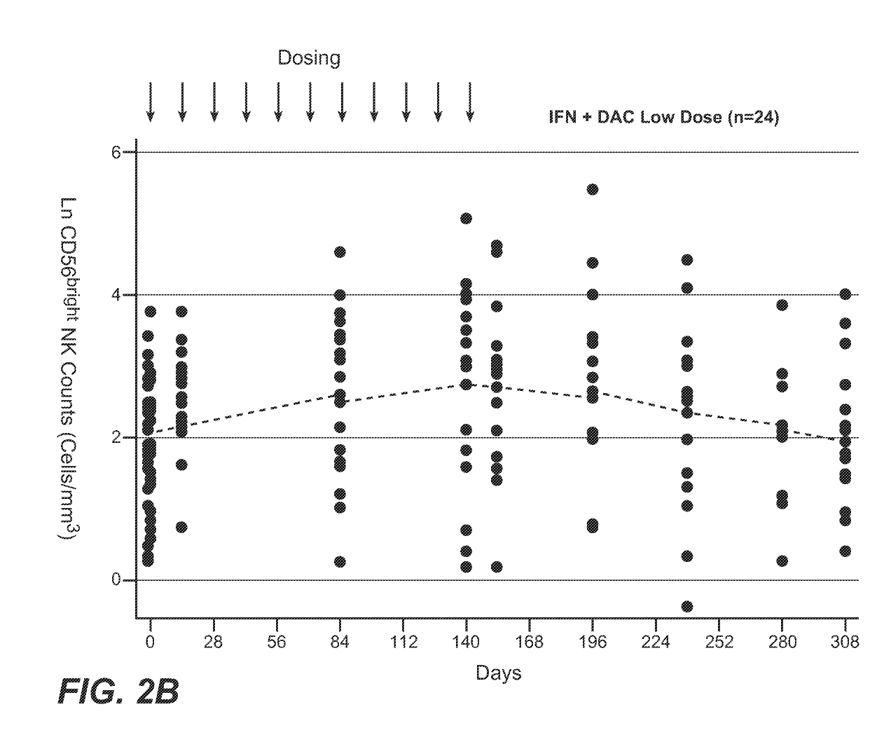

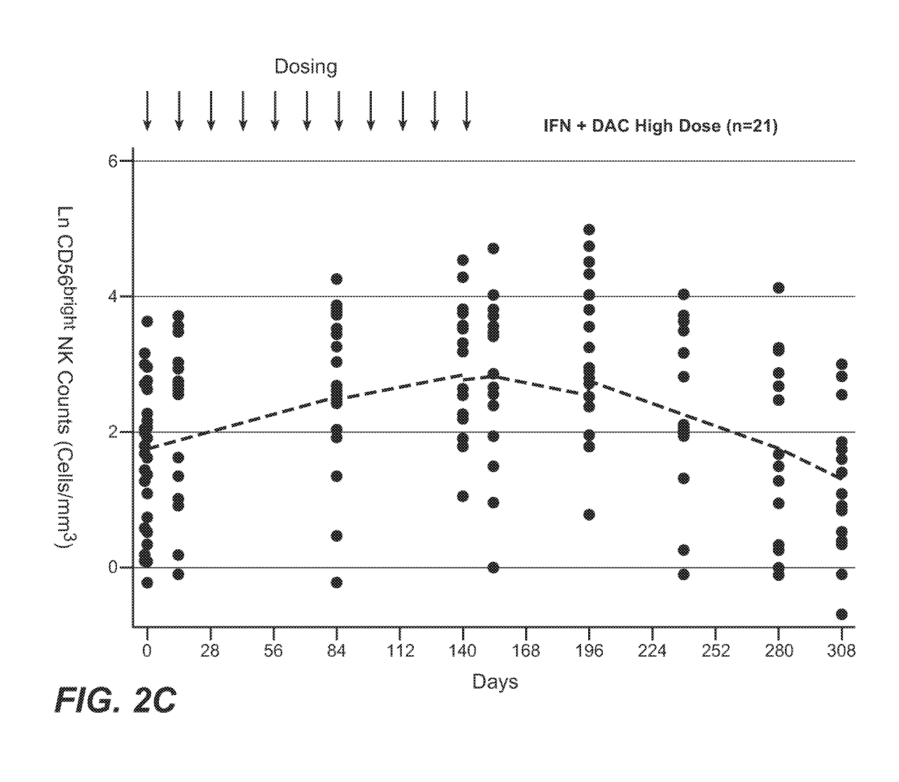

The methods described herein are based upon the discovery that CD3⁻CD56^{bright} NK cells (referred to herein as CD56^{bright} NK cells) expand rapidly in MS patients after these patients received their first dose of daclizumab (see FIGS. 2A-2C). Rapid expansion of CD56^{bright} NK cells was observed within 14 days of the first daclizumab dose. Rapid increases in absolute CD56^{bright} NK cells compared to baseline levels were observed for both low (1 mg/kg) and high level (2 mg/kg) daclizumab dosing groups (FIGS. 2B and 2C), whereas little or no change was observed for the placebo group (FIG. 2A). As shown in FIGS. 2B and 2C, expansion of CD56^{bright} NK cells in both dosing groups was observed after the first dose of daclizumab, reaching a plateau around day 140, and reversing following the cessation of daclizumab treatment. Expansion in CD56^{bright} NK cell levels correlated with reductions in Gd-CEL observed in daclizumab treated MS patients (FIG. 5).

In Table 1, the ability of CD56^{bright} NK cells to reduce Gd-CEL lesions in MS patients was evaluated as an independent variable of daclizumab exposure. An individual subject's CD56^{bright} NK cell count changes were ranked from those subjects with the least increase from baseline to those subjects with the greatest increase from baseline when treated with daclizumab. After ranking all daclizumab treated subjects by cell counts, the ranking was divided into roughly four equivalent size groups. Subjects with the least increase in CD56^{bright} NK cells from their baseline level (i.e. less than 25 percentile increase) were assigned to quartile 1 (Q1). Subjects with a 25 to 50 percentile increase were assigned to Q2. Subjects with a 50 to 75 percentile increase were assigned to Q3. Subjects with the greatest increase from baseline CD56^{bright} NK cell counts, i.e., greater than 75 percentile increase were assigned to Q4.

TABLE 1
Number of New or Enlarged Gd-CEL Lesions in
Placebo or Daclizumab Treated MS Patients

| Days | | | | | | |
|---|---|---|---|---|---|---|
| After | | Comparison group (placebo or CD56^{bright} expansion | | | | |
| First | | quartile by ranking) | | | | |
| Dose | | Placebo | Q1 | Q2 | Q3 | Q4 |
| 14 | Number of | 17 | 13 | 10 | 13 | 11 |
| | Subjects | | | | | |
| | Adjusted | 2.171 | 0.777 | 1.678 | 0.424 | 0.621 |
| | Mean | | | | | |
| | Number of | | | | | |
| | Lesions | | | | | |
| | P-Value | | 0.0992 | 0.6756 | 0.0236 | 0.0674 |
| | versus | | | | | |
| | placebo | | | | | |
| 84 | Number of | 17 | 12 | 12 | 12 | 11 |
| | Subjects | | | | | |
| | Adjusted | 2.149 | 1.210 | 1.189 | 0.684 | 0.221 |
| | Mean | | | | | |
| | Number of | | | | | |
| | Lesions | | | | | |
| | P-Value | | 0.3217 | 0.3366 | 0.0734 | 0.0106 |
| | versus | | | | | |
| | placebo | | | | | |

As shown in Table 1, CD56^{bright} NK cell expansion was detected at 14 days (following the first dose of daclizumab) and after 84 days (following three doses in the low dose group and six doses in the high dose group) in patients treated with daclizumab. The expansion in CD56^{bright} NK cells at both time points correlated with reductions in Gd-CEL observed in daclizumab treated MS patients. These observations suggest that changes in the level of CD56^{bright} NK cells following the first dose, second dose, third, dose, fourth dose, fifth dose and/or sixth dose of daclizumab would be a useful tool for monitoring the clinical response in MS patients to daclizumab or an anti-IL-2R antibody capable of inhibiting IL-2R signal transduction events.

Blood samples from MS patients can be analyzed for cell surface markers using flow cytometry (see, e.g., Bielekova, B., et al., 2006, Proc Natl Acad Sci USA, 103:5941-5946). Individual CD56^{bright} NK cells can be identified based on the characteristic staining pattern of CD16^{dim-to-negative} and CD56^{bright} using commercially available antibodies that bind preferentially to CD16 (for example, using clone 3G8 available from BD Bioscience, catalog number 557744 or an equivalent antibody clone) and CD56 (for example, using clone B159 available from BD Bioscience, catalog number 555518 or an equivalent antibody clone) and in combination with fluorescent activated cell sorting (FACS), the levels of CD56^{bright} NK expressing cells determined. Additionally, antibodies that bind preferentially to CD45 (for example, using clone HI30 available from BD Biosciences, catalog number 557748 or an equivalent antibody) can be used to enrich for all lymphocyte cells, and antibodies that bind preferentially to CD3 (for example, using clone SK7 available from BD Biosciences, catalog number 347347 or an equivalent antibody) can be used to eliminate T cells that express low levels of CD56 antigen, when optimizing the specific isolation of CD56^{bright} NK from other immune cells. The percent change in the number of CD56^{bright} NK cells following the first, second, third, fourth, fifth, and/or sixth dose of an anti-IL-2R antibody can be used to monitor the efficacy of an anti-IL-2R antibody.

The expansion of CD56^{bright} NK cells can be determined by absolute cell count, i.e., the number of cells per mm³ or mm², as a percent total of lymphocytes, or a percent total of a major immune subset, such as NK cells.

In some embodiments, other cell surface markers can be monitored to provide additional information regarding the clinical response in MS patients treated with an anti-IL-2R antibody. Additional cell surface markers include, but are not limited to, CD3, CD4, CD25, CD16, CD122, and CD8. Assays for the determination of these markers have been described, see, e.g., Bielekova, B., et al., 2006, Proc Natl Acad Sci USA, 103:5941-5946. For example, in some embodiments, the number of HLA-DR⁺CD4⁺ T cells can be analyzed and used to monitor the efficacy of an anti-IL-2R antibody (see, e.g., Sheridan, J P, et al., 2009, Neurology, 72 (11): A35-A35 Suppl).

The determination of the number of CD56^{bright} NK cells generally requires more than one sample be taken from a patient at selected times. The determination of sampling time is not critical to the methods described herein and can be selected by a medical practitioner based, in part on whether a patient has been treated with an anti-IL-2R antibody, or the length of time a patient has been treated with an anti-IL-2R antibody. Other factors that can affect sampling time include, but are not limited to, the length of time the patient has been treated for MS, which therapy(s) the patient has received prior to treatment with an anti-IL-2R antibody, and whether the patient is showing one or more of the following symptoms: increased relapse rate, an increase in the Expanded Disability Status Scale (EDSS) score, an increased number of new or enlarged Gd-CEL, and an increase in new or enlarged T2 MRI lesions. See, e.g., Perini et al., 2004, J Neurology, 251:305-309; Sorensen, et al., 2003, Lancet, 362:184-191; Pachner, 2003, Neurology, 61(Suppl 5):S2-S5; Perini et al., 2004, J Neurology, 251:305-309; and Farrell, et al., 2008, Multiple Sclerosis, 14:212-218.

In some embodiments, changes in the number of CD56^{bright} NK cells can be monitored before and within 14 days of the first dose of an anti-IL-2R antibody. By way of example, but not limitation, in some embodiments, the percent change in the number of CD56^{bright} NK cells can be determined by sampling prior to the first dose of an anti-IL-2R antibody and 14 days following the first dose of an anti-IL-2R antibody.

By way of another example, changes in the number of CD56^{bright} NK cells can be monitored prior to the first dose of an anti-IL-2R antibody and within 1, 2, 3, 4, 5, 6, 7, 8, 9, 10, 11, 12, 13 and 14 days following the first dose of an anti-IL-2R antibody, as well as at selected intervals thereafter (e.g., weekly, monthly, once every two months, once every three months, once every six months).

By way of another example, changes in the number of CD56^{bright} NK cells can be monitored prior to the first dose of an anti-IL-2R antibody and after a first dose, a second dose, a third dose, a fourth dose, a fifth dose, and/or a sixth dose of an anti-IL-2R antibody. The time at which the sample is taken after each dose is not critical, provided that the sampling is done before the next dose is administered.

Depending on the patient's medical history and symptoms, changes in the number of CD56^{bright} NK cells, can be monitored at two or more successive time points. For example, in some embodiments, changes in the number of CD56^{bright} NK cells can be monitored prior to the first dose of an anti-IL-2R antibody, after a first dose, and a second dose. By way of another example, changes in the number of CD56^{bright} NK cells can be monitored prior to the first dose of an anti-IL-2R antibody, and/or after a second dose of an anti-IL-2R antibody. Accordingly, any combination of sampling times prior to and following the first six dosing periods can be used to monitor the efficacy of an anti-IL-2R antibody in a patient diagnosed with multiple sclerosis.

The expansion or increase in the number of CD56^{bright} NK cells can be determined by comparing the number of CD56^{bright} NK cells following administration of an anti-IL-2R antibody to a reference. Typically, the reference represents a baseline number of CD56^{bright} cells prior to treatment with an anti-IL-2R antibody. The reference can be the number of CD56^{bright} NK cells detected in the patient prior to treatment with an anti-IL-2R antibody or an average number of CD56^{bright} cells detected in a population of individuals. For example, the mean number of CD56^{bright} cells/mm³ detected prior to treatment in the CHOICE study was 4.4±3.8 cells/mm³ in the placebo group, 8.8±8.7 cell/mm³ in the low dose group, and 7.7±8.9 cells/mm³ in the high dose group.

The average number of CD56^{bright} cells also can be obtained from a pool of multiple sclerosis patients prior to treatment with an anti-IL-2R antibody. Based on the results from the CHOICE, the reference number based on a pool of patients can vary from 4.4±3.8 cells/mm³ to 8.8±8.7 cell/mm³

Depending on the individual patient and the relapsing form of MS, the overall expansion or increase in the number of CD56^{bright} NK cells can vary from 0% to 500% following one or more doses of an anti-IL-2R antibody. For example in some embodiments, the number of CD56^{bright} NK cells can be increased by at least 25%, by at least 50%, by at least 75%, at least 100%, by at least 125%, by at least 150%, by at least 175%, by at least 200%, by at least 225%, by at least 250%, by at least 275%, by at least 300%, by at least 325%, by at least 350%, by at least 375%, by at least 400%, by at least by 425%, by at least 450%, by at least 475%, or by at least 500%.

As used herein, a “therapeutically effective dose” is a dose sufficient to prevent advancement, decrease the relapse rate, or reduce one or more of the symptoms associated with disease progression in multiple sclerosis.

For example, in some embodiments administration of a therapeutically effective dose of an anti-IL-2R antibody to a MS patient decreases the number of relapses by at least one that occur in a given time period, such as 1 year, in the treated patient. Relapses are typically assessed by history and physical examination defined as the appearance of a new symptom or worsening of an old symptom attributable to multiple sclerosis, accompanied by an appropriate new neurological abnormality or focal neurological dysfunction lasting at least 24 hours in the absence of fever, and preceded by stability or improvement for at least 30 days (see, e.g., Sorensen, et al., 2003, Lancet, 362: 1184-1191.

In other embodiments, administration of a therapeutically effective dose of an anti-IL-2R antibody to a MS patient decreases the number of lesions detected in the patient's brain. Magnetic Resonance Imaging (MRI) of the brain is an important tool for understanding the dynamic pathology of multiple sclerosis. T₂-weighted brain MRI defines lesions with high sensitivity in multiple sclerosis and is used as a measure of disease burden. However, such high sensitivity occurs at the expense of specificity, as T₂ signal changes can reflect areas of edema, demyelination, gliosis and axonal loss. Areas of Gd-CEL demonstrated on T₁-weighted brain MRI are believed to reflect underlying blood-brain barrier disruption from active perivascular inflammation. Such areas of enhancement are transient, typically lasting <1 month. Gd-CEL brain MRI is therefore used to assess disease activity. Most T₂-weighted (T2) lesions in the central white matter of subjects with multiple sclerosis begin with a variable period of Gd-CEL. Gd-CEL and T2 lesions represent stages of a single pathological process. Brain MRI is a standard technique for assessing Gd-CEL and T2 lesions and is routinely used to assess disease progression in MS (e.g., see Lee et al., Brain 122 (Pt 7):1211-2, 1999).

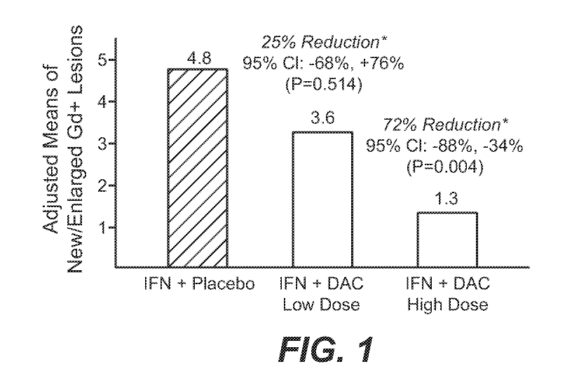

As shown in FIG. 1, treatment of MS patients with daclizumab reduced the mean number of new and enlarged Gd-CEL by 25% or more. Accordingly, in some embodiments a therapeutically effective dose of an anti-IL-2R antibody to a MS patient decreases the number of Gd-CEL detected in the patient's brain by approximately 25% to 80%. In some embodiments, the number of Gd-CEL detected in the patient's brain is decreased by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, and by at least 100%.

In other embodiments, administration of a therapeutically effective dose of an anti-IL-2R antibody to a MS patient decreases the number of T2 MRI lesions detected in the patient's brain.

In other embodiments, administration of a therapeutically effective dose of an anti-IL-2R antibody to a MS patient stabilizes a patient's disability progression as determined by the “Expanded Disability Status Scale (EDSS)” which can be used to rate neurological impairment in MS patients (Kurtzke, 1983, Neurology, 33-1444-52). The EDSS comprises 20 grades from 0 (normal) to 10 (death due to MS), progressing in a single-point step from 0 to 1 and in 0.5 point steps upward. The scores are based on a combination of functional-system scores, the patient's degree of mobility, need for walking assistance, or help in the activities of daily living. The functional-system scores measure function within individual neurological systems including visual, pyramidal, cerebellar, brainstem, sensory, bowel and bladder, cerebral and other functions.

In other embodiments, administration of a therapeutically effective dose of an anti-IL-2R antibody to an IFN-beta NAb positive MS patient reduces a patient's disability score by 10% to 75%. For example in some embodiments, a patient's disability score can be reduced by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, or by at least 75%.

In some embodiments, treatment of an MS patient with an anti-IL-2R antibody may be insufficient to prevent permanent disabilities or delay disease progression. MS patients that are responding poorly to therapy with an anti-IL-2R antibody generally have a higher mean relapse rate, a higher risk of experiencing a second relapse, a higher risk of having a sustained progression of >1 on EDSS, and a lower probability of being relapse free (Malucchi, et al., 2004, Neurology, 62: 2031-2037). Accordingly, a number of clinical endpoints can be used to determine whether a patient is responding to treatment with an anti-IL-2R antibody including the frequency and rate of relapse, a 1 point or greater increase in the Expanded Disability Status Scale (EDSS) score, an increase in the number of Gd-CEL, and/or an increase in the number of T2 MRI lesions.

The data required to determine clinical endpoints can be collected at the start of the anti-IL-2R treatment and/or during follow-up visits. In some embodiments, MS patients that are responding poorly to an anti-IL-2R antibody can be treated with additional agents. For example, in some embodiments, one or more anti-IL-2R antibodies can be administered to a MS patient. In other embodiments, an anti-IL-2R antibody can be administered in combination with another MS therapy, such as an IFN-beta product. Examples of suitable IFN-beta products include, but are not limited to, one of the three IFN-beta products that have been approved: IFN-beta-1b (Betaferon®, Schering AG, Berlin, Germany), IFN-beta-1a (Avonex®, Biogen Idec, Cambridge Mass.), and IFN-beta-1a (Rebif®, Ares-Serono, Geneva, Switzerland). Non-limiting examples of other marketed drugs used to treat MS that may be used in combination with an anti-IL-2R treatment include glatiramer acetate (e.g., Copaxone®, Teva Pharmaceutical Industries, Ltd., Israel), natalizumab, cladribine, corticosteroids, riluzole, azathioprine, cyclophosphamide, methotrexate, and mitoxantrone.

In some embodiments, CD56^{bright} NK cell expansion can be stimulated in MS patients that are not responding or responding poorly to treatment with an anti-IL-2R antibody by administering low-dose recombinant interleukin-2 (rIL-2). rIL-2 has been shown to increase absolute NK cell numbers, with the most pronounced increase occurring within the CD3⁻CD56^{bright} NK cell subset (Khatri, V. P., et al., 1997, Cancer J. Sci. Am., 3:S128-S136). In some embodiments, rIL-2 can be administered in combination with an anti-IL-2R antibody to MS patients exhibiting no change or a decrease in the number of CD56^{bright} NK cells following treatment with an anti-IL-2R antibody. In other embodiments, CD56^{bright} NK cell expansion can be stimulated in MS patients assigned to quartiles Q1, Q2, Q3, or Q4 by administering rIL-2 in combination with an anti-IL-2R antibody. In yet other embodiments, rIL-2 can be administered in combination with an anti-IL-2R antibody to MS patients exhibit little increase in CD56^{bright} NK cells from their baseline level (e.g., less than 25 percentile increase, assigned to Q1) or to MS patients exhibiting a modest increase CD56^{bright} NK cells from their baseline level (e.g., 25 to 50 percentile increase, assigned to Q2).

Effective doses of rIL-2 can vary depending upon a variety of factors, including, means of administration, physiological state of the patient, administration of other medication, and whether the treatment is prophylactic or therapeutic. Treatment dosages can be optimized, for example, by determining the maximum tolerated dose (MTD) for a given means of administration (e.g., intravenously or subcutaneously) and dosing frequency (e.g., daily, weekly, monthly). In some embodiments, rIL-2 can be administered subcutaneously at doses ranging from 0.1×10⁶ to 2.0×10⁶ IU/m²/day (Khatri, V. P., et al., 1997, Cancer J. Sci. Am., 3:S128-S136). rIL-2 can be administered prior to or at the same time as a therapeutically effective dose of an anti-IL-2R antibody. Therapeutically effective doses of an anti-IL-2R antibody are described herein. In other embodiments, dosage and/or dosing frequency can be optimized to maintain the number of CD56^{bright} NK cells at a level sufficient to reduce the mean number of new and enlarged Gd-CEL detected in the patient's brain by approximately 25% to 80% when rIL-2 is administered in combination with an anti-IL-2R antibody.

In some embodiments, the interval of dosing can be adjusted. For example, but not by way of limitation, if the standard dose of an anti-IL-2R antibody is 150 mg monthly and a rapid expansion in CD56^{bright} cells is observed in the treated patient, the interval of dosing can be increased from monthly to every two months or longer. In other embodiments, if no change or a decrease in the expansion of CD56^{bright} cells is observed in the treated patient, the interval of dosing can be decreased from 150 mg monthly to 150 mg biweekly or weekly.

In other embodiments, the dosage can be adjusted. For example, but not by way of limitation, if the standard dose of an anti-IL-2R antibody is 150 mg monthly and no change, or a decrease in the expansion of CD56^{bright} cells is observed in the treated patient, the dose can be increased to 200 mg, 300 mg, 400 mg, or up to 500 mg monthly. By way of another example, if a standard dose of an anti-IL-2R antibody is 150 mg monthly and a rapid expansion in CD56^{bright} cells is observed, the dose can be decreased to 100 mg, or 50 mg monthly.

Changes in dosage, the interval of dosing, and the use of additional therapeutic agents can be used in combination with an anti-IL2R antibody to increase the efficacy of the anti-IL-2R antibody in a patient diagnosed with multiple sclerosis.

MS patients suitable for treatment with an anti-IL-2R antibody typically have been diagnosed with a relapsing form of multiple sclerosis including relapsing-remitting multiple sclerosis, secondary progressive multiple sclerosis, progressive relapsing multiple sclerosis and worsening relapsing multiple sclerosis. By “relapsing-remitting multiple sclerosis” herein is meant a clinical course of MS that is characterized by clearly defined, acute attacks with full or partial recovery and no disease progression between attacks. By “secondary-progressive multiple sclerosis” herein is meant a clinical course of MS that initially is relapsing-remitting, and then becomes progressive at a variable rate, possibly with an occasional relapse and minor remission. By “progressive relapsing multiple sclerosis” herein is meant a clinical course of MS that is progressive from the onset, punctuated by relapses. There is significant recovery immediately following a relapse, but between relapses there is a gradual worsening of disease progression. By “worsening relapsing multiple sclerosis” herein is meant a clinical course of MS with unpredictable relapses of symptoms, from which people do not return to normal and do not recover fully.

In some embodiments, the anti-IL-2 receptor antibody is daclizumab. The recombinant genes encoding daclizumab are a composite of human (about 90%) and murine (about 10%) antibody sequences. The donor murine anti-Tac antibody is an IgG2a monoclonal antibody that specifically binds the IL-2R Tac protein and inhibits IL-2-mediated biologic responses of lymphoid cells. The murine anti-Tac antibody was “humanized” by combining the complementarity-determining regions and other selected residues of the murine anti-Tac antibody with the framework and constant regions of the human IgG1 antibody. The humanized anti-Tac antibody daclizumab is described and its sequence is set forth in U.S. Pat. No. 5,530,101, see SEQ ID NO: 5 and SEQ ID NO: 7 for the heavy and light chain variable regions respectively. SEQ ID NOS: 5 and 7 of U.S. Pat. No. 5,530,101 are disclosed as SEQ ID NOS: 1 and 2 respectively in the sequence listing filed herewith. U.S. Pat. No. 5,530,101 and Queen et al., Proc. Natl. Acad. Sci. 86:1029-1033, 1989 are both incorporated by reference herein in their entirety.

Daclizumab has been approved by the U.S. Food and Drug Administration (FDA) for the prophylaxis of acute organ rejection in subjects receiving renal transplants, as part of an immunosuppressive regimen that includes cyclosporine and corticosteroids, and is marketed by Roche as ZENAPAX®. Daclizumab also has been shown to be active in the treatment of human T cell lymphotrophic virus type 1 associated myelopathy/topical spastic paraparesis (HAM/TSP, see Lehky et al., Ann. Neuro., 44:942-947, 1998). The use of daclizumab to treat posterior autoimmune uveitis has also been described (see Nussenblatt et al., Proc. Natl. Acad. Sci., 96:7462-7466, 1999).

Antibodies that bind the same (or overlapping) epitope as daclizumab can be used in the methods disclosed herein. In some embodiments, the antibody will have at least 90%, at least 95%, at least 98%, or at least 99% sequence identity with daclizumab. The antibody can be of any isotype, including but not limited to, IgG1, IgG2, IgG3 and IgG4.

In some embodiments, the antibody is basiliximab, marketed as Simulect® by Novartis Pharma AG. Basiliximab is a chimeric (murine/human) antibody, produced by recombinant DNA technology that functions as an immunosuppressive agent, specifically binding to and blocking the alpha chain of the IL-2R on the surface of activated T-lymphocytes.

Anti-IL-2R antibodies can be administered parenterally, i.e., subcutaneously, intramuscularly, intravenously, intranasally, transdermally, or by means of a needle-free injection device. The compositions for parenteral administration will commonly include a solution of an anti-IL-2R antibody in a pharmaceutically acceptable carrier. Pharmaceutically-acceptable, nontoxic carriers or diluents are defined as vehicles commonly used to formulate pharmaceutical compositions for animal or human administration. See, for example, Remington: The Science and Practice of Pharmacy, A. R. Gennaro, 20th Edition, 2001, Lippincott Williams & Wilkins, Baltimore, Md., for a description of compositions and formulations suitable for pharmaceutical delivery of the anti-IL-2R antibodies disclosed herein. See U.S. Pat. Appl. Pub. Nos. 2003/0138417 and 2006/0029599 for a description of liquid and lyophilized formulations suitable for the pharmaceutical delivery of daclizumab.

Methods for preparing pharmaceutical compositions are known to those skilled in the art (see Remington: The Science and Practice of Pharmacy, supra). In addition, the pharmaceutical composition or formulation can include other carriers, adjuvants, or nontoxic, non-therapeutic, nonimmunogenic stabilizers and the like. Effective amounts of such diluent or carrier will be those amounts that are effective to obtain a pharmaceutically acceptable formulation in terms of solubility of components, or biological activity.

The concentration of antibody in the formulations can vary widely, i.e., from less than about 0.5%, usually at or at least about 1% to as much as 15 or 20% by weight or from 1 mg/mL to 100 mg/mL. The concentration is selected primarily based on fluid volumes, viscosities, etc., in accordance with the particular mode of administration selected.

Generally a suitable therapeutic dose of daclizumab is about 0.5 milligram per kilogram (mg/kg) to about 5 mg/kg, such as a dose of about 0.5 mg/kg, of about 1 mg/kg, about 1.5 mg/kg, about 2 mg/kg, about 2.5 mg/kg, about 3.0 mg/kg, about 3.5 mg/kg, about 4.0 mg/kg, about 4.5 mg/kg, or about 5.0 mg/kg administered intravenously or subcutaneously. Unit dosage forms are also possible, for example 50 mg, 100 mg, 150 mg, 200 mg, 300 mg, 400 mg, or up to 500 mg per dose.

Other dosages can be used to obtain serum levels of 2 to 5 μg/mL which are necessary for saturation of the Tac subunit of the IL-2 receptor to block the responses of activated T lymphocytes. Higher levels such as approximately 5 to 40 μg/mL, may be necessary for clinical efficacy. One of skill in the art will be able to construct an administration regimen to keep serum levels within the 2 to 40 μg/mL range.

In some embodiments, daclizumab is administered monthly in a unit dosage form of 150 mg.

Doses of basiliximab are likely to be lower, for example 0.25 mg/kg to 1 mg/kg, e.g., 0.5 mg/kg, or unit doses of 10, 20, 40, 50 or 100 mg, due to the higher affinity of basiliximab for the IL-2R target. The general principle of keeping the IL-2 receptor saturated can be used to guide the choice of dose levels of other IL-2R antibodies.

Single or multiple administrations of anti-IL-2R antibodies can be carried out with dosages and frequency of administration selected by the treating physician. Generally, multiple doses are administered. For example, multiple administration of daclizumab or other anti-IL-2R antibodies can be utilized, such as administration monthly, bimonthly, every 6 weeks, every other week, weekly or twice per week.

### 6. EXAMPLES

### Example 1

### CHOICE Study

The CHOICE study was a Phase 2, randomized, double-blinded, placebo-controlled, multi-center study of subcutaneous (SC) daclizumab added to interferon (IFN)-beta in the treatment of active, relapsing forms of MS. Results from the CHOICE study confirmed that daclizumab at 2 mg/kg every two weeks significantly decreased the number of new Gd-CEL in patients who have active, relapsing forms of MS on concurrent IFN-beta therapy (Montalban, X. et al., Multiple Sclerosis, 13: S18-S18 Suppl. 2 OCT 2007; and, Kaufman, M. D., et. al., Neurology, 70 (11): A220-A220 Suppl. 1 Mar. 11, 2008). A smaller decrease in new or enlarging Gd-CEL was observed for those study subjects receiving 1 mg/kg daclizumab every four weeks.

A patient was enrolled in the study once he or she was randomized. Enrolled patients remained on their baseline IFN-beta regimen and were randomized in a 1:1:1 ratio to one of the following 3 treatment arms (see Table 2).

TABLE 2

| | | No. Total | No. |
|---|---|---|---|
| Treatment Arm¹ | Dose Level and Frequency | Dosing Visits | Patients |
| A (High Dose)² | Daclizumab SC: 2 mg/kg | 11 | 55 |
| | q2 weeks × 11 doses | | |
| B (Low Dose)³ | Daclizumab SC: 1 mg/kg | 11 | 55 |
| | q4 weeks × 6 doses | | |
| C (Placebo)⁴ | Placebo SC: q2 weeks × | 11 | 55 |
| | 11 doses | | |

¹All patients continue on prior regimen of IFN-beta SC/IM for the duration of the study.
²Patients in Arm A (high dose) receive 2 SC injections (2 daclizumab 1 mg/kg) for 11 dosing visits. Maximum dose daclizumab per dosing visit = 200 mg.
³Patients in Arm B (low dose) receive 2 SC injections (1 daclizumab 1 mg/kg, 1 placebo) for 6 dosing visits, alternating with 2 SC injections (2 placebo) for 5 dosing visits. Maximum dose daclizumab per daclizumab dosing visit = 100 mg.
⁴Patients in Arm C (placebo) receive 2 SC injections (2 placebo) for 11 dosing visits.

The screening period was up to 3 weeks. The treatment period was designated as 24 weeks (6 months, through Day 168) in order to include 4 weeks subsequent to the last dose of blinded study drug (Dose No. 11, which occurs at Visit No. 14, Day 140). After the treatment period, patients were followed for a total of 48 weeks (12 months) and continued IFN beta therapy for at least 5 months of this period. Total maximum time on study for each patient was approximately 18 months.

Evaluations of a given patient by EDSS and Multiple Sclerosis Functional Composite, version 3 (MSFC-3) were performed by a clinician who was not involved in the patient's treatment and was designated an “evaluating clinician.” All other assessments of the patient were under the purview of the clinician in charge of the patient's treatment (treating clinician). The MSFC-3 includes quantitative tests of: (1) Leg function/ambulation—Timed 25-foot walk (T25FW); (2) Arm function—9-Hole Peg Test (9HPT), and (3) Cognition—Paced Auditory Serial Addition Test with 3-second interstimulus intervals (PASAT3) (Cutter et al., 1999, Brain, 122(Pt 5):871-882).

Preliminary eligibility for the CHOICE study was established by history, chart inspection, and routine evaluations. During the treatment and follow up period, a number of procedures and evaluations were performed on the subjects at specified days including, but not limited to, MRI, EDSS, MSFC-3, physical exams, symptom directed physical exams, hematology/serum chemistry (e.g., for determination of pharmacokinetic assessment and anti-DAC antibodies), and blood draws for pharmacodynamic assessments and IFN-beta NAbs.

Daclizumab drug substance manufactured by PDL BioPharma, Inc. (Redwood City, Calif.) for subcutaneous delivery, was supplied in single-use vials containing 100 mg of daclizumab in 1.0 mL of 40 mM sodium succinate, 100 mM sodium chloride, 0.03% polysorbate 80, pH 6.0. Placebo was supplied in single-use vials as an isotonic solution in matching vials containing 40 mM sodium succinate, 6% sucrose, 0.03% polysorbate 80, pH 6.0.

### Example 2

### Expansion of CD56^{bright} NK Cells in MS Patients Treated with Daclizumab

Methods: CD56^{bright} NK cell counts were obtained by performing blinded, flow cytometric analysis (FACS) on banked peripheral blood mononuclear cells (PBMC) collected at 10 time points during the CHOICE study.

Individual CD56^{bright} NK cells were identified based on the characteristic staining pattern of CD16^{dim-to-negative} and CD56^{bright}. Conversion of banked specimen FACS results into cell count per unit volume of blood were possible by factoring percentages against absolute lymphocyte cell count data obtained from freshly prepared whole blood specimens analyzed by TruCOUNT™ at the time of collection.

Parametric (paired t test) and non-parametric (Wilcoxon) analyses were performed comparing levels of immune subset cell counts between dosing groups.

Individual DAC exposure characteristics from a subset of subjects, including steady state trough (C_{ss,min}), and AUCₛₛ (calculated area under the change from baseline-time curve), were used separately as predictors to model changes from baseline level in individual CD56^{bright} NK cells changes over time.

Locally weighted scatterplot smoothing (Lowess) curve fitting analysis was used to model the CD56^{bright} NK cell counts by treatment group.

Negative binomial regression model adjusted for the mean number of Gd-CEL lesions at baseline as determined using MRI was used to estimate P-values.

Results: FIGS. 2A-2C are Lowess plots of absolute CD56^{bright} NK cell counts over time. Rapid increases in absolute CD56^{bright} NK cells compared to baseline levels were observed for both daclizumab dosing groups (FIGS. 2B-2C), whereas little or no change was observed for the placebo group (FIG. 2A). Analysis was performed using FACS, blinded to treatment group. Expansion of CD56^{bright} NK cells in both dosing groups reached a plateau and reversed following daclizumab treatment discontinuation and subsequent de-saturation of the CD25 antigen on CD25 expressing immune subsets beginning approximately Day 196 (data not shown).

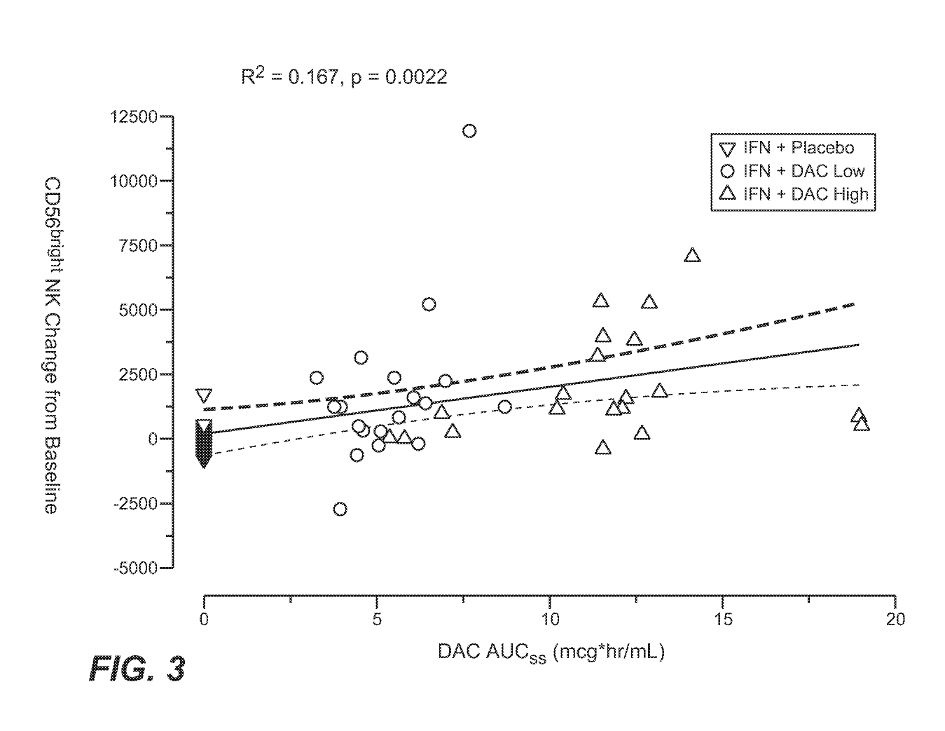

A statistically significant (p=0.0022) positive linear relationship was observed between each individual's steady state exposure to daclizumab and their increase in absolute CD56^{bright} NK cell counts when change from baseline level of absolute CD56^{bright} NK cell counts were evaluated as area under the curve (AUC) values during the dosing period (FIG. 3). Included in the analysis were results obtained for all subjects with at least one baseline and one post-dose pharmacokinetic (PK) and pharmacodynamic (PD) assay result when treated with IFN+placebo (n=15), IFN+daclizumab low dose (n=20) and IFN+daclizumab high dose (n=20). Results of linear correlation and 95% confidence interval are shown.

As shown in Table 3, significant changes in CD56^{bright} NK, but not lymphocyte populations, were concomitant with daclizumab treatment. Interquartile ranges (IQR) and P value from Wilcoxon comparison to placebo group absolute cell count levels at baseline, Week 20, and Week 44 are shown. Median CD56^{bright} NK cells cell counts at baseline for the IFN+daclizumab low dose group were approximately 2-fold higher compared to the IFN+daclizumab high dose group, and both daclizumab dose groups were associated with 7-8 fold increase in comparison to IFN+Placebo levels at Day 140.

TABLE 3
Changes in Lymphocyte Populations Following Daclizumab
(DAC) Administration and Withdrawal

| Immune | | Baseline cells/mm³ | Week 20 cells/mm³ | Week 44 cells/mm³ |
|---|---|---|---|---|
| Subset | Treatment | Median (IQR) P value | Median (IQR) P value | Median (IQR) P value |
| CD4T | Placebo/IFN | 735 (568-867) | 604 (510-733) | 687 (535-798) |
| | DAC Low/IFN | 789 (471-1012) 0.45 | 780 (581-864) 0.079 | 784 (620-988) 0.27 |
| | DAC High/IFN | 652 (560-951) 0.89 | 615 (429-816) 0.81 | 681 (438-834) 0.97 |
| CD8T | Placebo/IFN | 249 (153-330) | 232 (130-265) | 258 (190-365) |
| | DAC Low/IFN | 275 (196-97) 0.30 | 265 (230-402) 0.064 | 312 (204-433) 0.36 |
| | DAC High/IFN | 269 (180-400) 0.61 | 241 (146-327) 0.29 | 256 (171-342) 0.65 |
| NK (all) | Placebo/IFN | 103 (86-121) | 114 (106-133) | 114 (87-143) |
| | DAC Low/IFN | 87 (69-125) 0.31 | 125 (99-162) 0.41 | 100 (67-139) 0.61 |
| | DAC High/IFN | 96 (74-109) 0.23 | 142 (88-175) 0.15 | 105 (42-157) 0.71 |
| CD56^{bright} | Placebo/IFN | 2.9 (1.9-6.5) | 2.7 (1.5-4.3) | 2.6 (1.4-8.5) |
| NK | DAC Low/IFN | 6.5 (4.0-11.3) 0.03 | 19.8 (6.2-40.1) 0.002 | 6.5 (4.2-10.9) 0.13 |
| | DAC High/IFN | 4.6 (1.9-9.3) 0.33 | 24.9 (9.3-35.9) <0.001 | 3.6 (1.7-6.4) 0.59 |

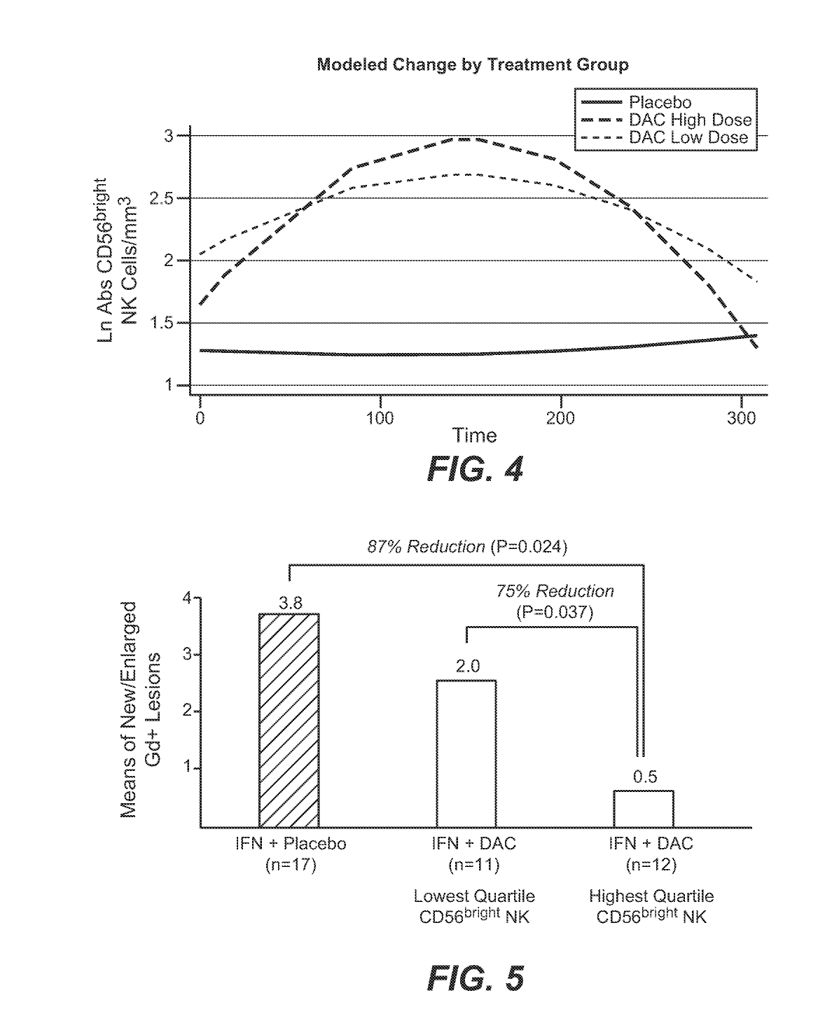

The dose-dependency of daclizumab to expand CD56^{bright} NK cells was evaluated using a mixed-effects linear regression model that assumed parabolic growth based on Lowess data and model fit. As shown in FIG. 4, the IFN+high daclizumab (DAC) dose resulted in significantly greater expansion compared to IFN+low daclizumab (DAC) dose (treatment X Time, p=0.008 and treatment X Time2, p=0.003). A significantly greater expansion was also observed for IFN in combination with daclizumab at either dose compared to IFN+placebo (treatment X Time, <=0.001 and treatment X Time2, <=0.001).

FIG. 5 depicts new and enlarged Gd-CEL lesions by quartile of CD56^{bright} NK expansion when measured at the last daclizumab dose (week 20). A statistically significant relationship (p=0.037) was also observed within all daclizumab treated subjects, irrespective of daclizumab dosing group, when Gd-CEL lesions were compared for the highest quartile subjects verses the lowest quartile subjects in terms of CD56^{bright} NK cell counts at week 20. Analysis includes 65 PD sub-study subjects.

Taken together, these results demonstrate that daclizumab causes a robust, dose-dependent expansion of CD56^{bright} NK cells in peripheral blood, and the expansion of these immune cells is associated with reductions in Gd-CEL lesions. These findings support a role for CD56^{bright} NK cells as an efficacy biomarker and effect mediator of daclizumab in MS.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

1-45. (canceled)

46. A method of monitoring the efficacy of an anti-IL-2R antibody in a patient diagnosed with multiple sclerosis comprising, determining the level of CD⁵⁶bright NK cells in blood samples obtained from a patient diagnosed with multiple sclerosis before and after administration of at least a first dose of an anti-IL-2R antibody, wherein an increase in the level of CD⁵⁶bright NK cells in the patient following administration of said dose of the anti-IL-2R antibody indicates that the anti-IL-2R antibody is effective in ameliorating at least one symptom of multiple sclerosis in the treated patient.

47. The method according to claim 46, wherein the blood sample is collected within 14 days after administration of a first dose of the anti-IL-2R antibody.

48. The method according to claim 46, wherein the blood sample is collected within one month after administration of a first dose of the anti-IL-2R antibody.

49. The method according to claim 46, wherein the antibody that specifically binds the interleukin 2 receptor is a humanized antibody.

50. The method according to claim 46, wherein the anti-IL-2R antibody specifically binds to the alpha subunit of the human high-affinity interleukin-2 receptor and inhibits IL-2 signaling.

51. The method of claim 50, wherein the anti-IL-2R antibody is a humanized antibody.

52. The method of claim 51, wherein the humanized antibody is daclizumab.

53. The method of claim 52, wherein daclizumab is administered at a dose of 150 mg.

54. The method of claim 52, wherein daclizumab is administered intravenously, subcutaneously, intramuscularly, intranasally, or transdermally.

55. The method of claim 52, wherein daclizumab is administered at least monthly.

56. The method according to claim 46, wherein the subject has a relapsing form of multiple sclerosis.

57. A method of monitoring the response to an anti-IL-2R antibody in a patient diagnosed with multiple sclerosis comprising, comparing the level of CD⁵⁶bright NK cells in a patient diagnosed with multiple sclerosis between 14 days and one month following administration of a first dose of an anti-IL-2R antibody to a reference number of CD⁵⁶bright NK cells, wherein an increase in the level of CD56bright NK cells following administration of a first dose of the anti-IL-2R antibody compared to the reference number of CD⁵⁶bright NK cells indicates the effectiveness of the anti-IL-2R antibody treatment in ameliorating at least one symptom of multiple sclerosis in the treated patient.

58. The method according to claim 57, wherein the reference number is 4.4±3.8 to 8.8±8.7 CD⁵⁶bright NK cells/mm³.

59. The method of claim 57, wherein the reference number is based on the number of CD⁵⁶bright NK cells/mm³ in a blood sample obtained from the subject prior to treatment with the anti-IL-2R antibody.

60. The method according to claim 57, wherein the antibody that specifically binds the interleukin 2 receptor is a humanized antibody.

61. The method according to claim 57, wherein the anti-IL-2R antibody specifically binds to the alpha subunit of the human high-affinity interleukin-2 receptor and inhibits IL-2 signaling.

62. The method of claim 61, wherein the anti-IL-2R antibody is a humanized antibody.

63. The method of claim 61, wherein the humanized antibody is daclizumab.

64. The method of claim 63, wherein daclizumab is administered at a dose of 150 mg.

65. The method of claim 63, wherein daclizumab is administered intravenously, subcutaneously, intramuscularly, intranasally, or transdermally.

66. The method of claim 63, wherein daclizumab is administered at least monthly.

67. The method according to claim 57, wherein the subject has a relapsing form of multiple sclerosis.

68. A method of monitoring the efficacy of an anti-IL-2R antibody in a patient diagnosed with multiple sclerosis, comprising comparing the level of CD⁵⁶bright cells in blood samples collected from a patient diagnosed with multiple sclerosis before and after administration of at least a first dose of an anti-IL-2R antibody to determine if the level of CD⁵⁶bright cells has increased, decreased, or not changed following treatment with the anti-IL-2R antibody.

69. The method according to claim 68, in which the blood sample is collected from the patient 14 days after the first dose.

70. The method according to claim 68, in which the blood sample is collected from the patient one month after the first dose.

71. The method according to claim 68, in which the change in the level of CD⁵⁶bright cells in the treated patient is increased by at least 25% to 500%.

72. The method according to claim 68, in which an increase in the number of CD⁵⁶bright cells in the treated patient indicates that the anti-IL-2R antibody is effective in ameliorating at least one symptom of multiple sclerosis in the treated patient.

73. The method according to claim 68, wherein no change in the level of CD⁵⁶bright cells in the treated patient is detected and treatment with the anti-IL-2R antibody is supplemented with one or more additional agents or terminated.

74. The method according to claim 68, wherein a decrease in the level of CD⁵⁶bright cells in the treated patient is detected and treatment with the anti-IL-2R antibody is supplemented with one or more additional agents or terminated.

75. The method according to claim 68, wherein a change in the level of CD⁵⁶bright NK cells in the treated patient is detected and the dosage or interval of dosing with the anti-IL-2R antibody is increased or decreased according to the number of CD⁵⁶bright NK cells detected.
